(19) United States
(12) Patent Application Publication
(10) Pub. No.: US 2008/0108870 A1
(43) Pub. Date: May 8, 2008

(54) Apparatus and method for stabilizing an image from an endoscopic camera

(76) Inventors: Bruce E. Wiita, Jupitter, FL (US); Gregory D. Witta, Jupiter, FL (US)

Correspondence Address: NORMAN FRIEDLAND, 2855 PGA BOULEVARD, SUITE 200, PALM BEACH GARDENS, FL, 33410, US

(21) Appl. No.: 11/593,626

(22) Filed: Nov. 6, 2006

(51) Int. Cl. A61B 1/05

(52) U.S. Cl. 600/112

(57) ABSTRACT

A video camera unit used with an endoscope is provided with sensors sensing an ambient field to detect rotation of the camera unit about an axis causing rotation of an image generated from the output signal of the camera unit. This output signal is modified to produce a stabilized video signal, producing an image held at a preferred angle. In one embodiment, two ambient fields, such as gravity and a magnetic field are alternately used.

### BACKGROUND OF THE INVENTION

1. Field of the Invention

This invention relates to stabilization of an image produced from the output signal of a video camera used with an endoscope, and, more particularly, to eliminating the rotation of the displayed image caused by rotation of the video camera.

2. Summary of the Background Art

A video camera is often used with, or as a part of, an endoscope in diagnostic or surgical procedures, with images generated from the video camera signals being displayed on the screen of a display monitor for use by the surgeon and other individuals during the procedures. However, as the endoscope is maneuvered through various channels within the patient's body, and as the endoscope and the video camera are further maneuvered to place a particular tissue structure within the field of view of the video camera, the camera is often rotated about its optical axis, causing the image displayed on the monitor screen to be similarly rotated. Such rotation of the displayed image can make it difficult to interpret the information displayed on the screen.

The patent literature includes descriptions of a number of methods for preventing such rotation of the displayed image in one of three ways. First, the rotation of the image sensor may be prevented, or at least minimized. Second, the optical image may be rotated by optical means before it reaches the sensing device. Third, the image derived from the video cameral signals may be electronically rotated within a processor before it is displayed.

For example, as described in European Patent No. 0501088B1, the video camera can be mounted to freely rotate about its optical axis, with a weight held away from its optical axis by a rod preserving the angular position of the camera despite rotation of the endoscope, as long as the optical axis of the camera is not vertical or close to vertical.

U.S. Pat. No. 5,005,943 describes a rigid video endoscope having optical means for rotating the image between the image producing optics of the endoscope and the video camera.

U.S. Pat. No. 6,097,423 describes an endoscope in which an accelerometer generates a signal indicating the local vertical, which is used to rotate an image sensor aligned with the optical axis of the endoscope to maintain a desired orientation of an image displayed on a monitor.

U.S. Pat. No. 6,471,637 describes an endoscope having an inertial sensor to sense rotations of the received image about the optical axis of the endoscope, with the output of the inertial sensor being used to rotate either the image or the image sensor. To sense rotations, the inertial sensor can be a gyroscope or a pair of accelerometers. In the case of rotation of the image obtained with the image sensor, the image is rotated within a microprocessor for subsequent viewing on a video display, with signal processing compensatory rotation of the displayed image as an operator of the endoscope moves the instrument about.

U.S. Pat. App. Pub. No. 2005/015260 A1 describes an endoscope having three accelerometers, responsive to gravity, mounted on its housing, with each accelerometer measuring a component of gravity along a particular measurement axis. The accelerometers provide pulse-width-modulated signals to a processor, which converts each signal into a gravitational force measurement. Changes in the gravitational force measurements from the accelerometers are then related to rotation of the endoscope. Calculations within the processor include factors to account for endoscope roll, endoscope pitch, and endoscope viewing direction.

Japanese Pat, App. No. 06269403 A describes an endoscope including an electronic abdominal mirror and two magnetic coils, which are used to calculate the rotation of the main body of the endoscope by detecting the strength of a physical parameter, such as a magnetic field or a gravitational field within a space occupied by the main body of the endoscope. An image rotation correcting means is additionally provided to rotate the image formed by a video camera so that the image on a display monitor is maintained in an erect normal state3 corresponding to the rotation of an abdominal mirror.

### SUMMARY OF THE INVENTION

A method is provided for stabilizing a displayed image formed from an output signal of a camera unit used with an endoscope. The method includes periodically determining a value of an angle of rotation of the camera unit about an axis of image rotation relative to a direction of an ambient field by an angle determining process. The axis of image rotation is defined as an axis about which rotation of the camera unit causes rotation of an image formed from the output signal of the camera unit. After a value of the output signal is set as a preferred angle of rotation, the output signal of the camera unit is processed to form a stabilized video signal by an image stabilizing process causing the displayed image to be rotated through a correction angle derived by calculating a difference between the most recently determined value of the angle of rotation and the preferred angle of rotation. A display unit is driven with the stabilized video signal.

It is understood that repeatedly returning a displayed image to the previous angle is equivalent to holding the image at a saved preferred angle.

According to one aspect of the invention, the angle determining process includes:

receiving a first signal representing a level of the ambient field measured in a first direction relative to the camera unit, with the first direction being perpendicular to the axis of image rotation;

receiving a second signal representing a level of the ambient field measured in a second direction relative to the camera unit, with the second direction being perpendicular to the axis of image rotating and to the first direction;

determining a tangent of the angle of rotation by dividing a value representing a level of the first signal by a value representing a level of the second signal; and

determining a quadrant of the angle as a function of the signs of the first and second signals.

According to another aspect of the invention, the image stabilizing process includes:

writing pixel data derived from the output signal of the camera unit, representing light intensities measured at an image sensor within the camera unit in a first plurality of intersections between a first plurality of horizontal lines and a first plurality of vertical lines to a corresponding plurality of locations within a data buffer;

generating a sequence of addresses identifying locations within the corresponding plurality of locations within the data buffer, wherein the sequence of addresses identifies locations storing data representing light intensities measured nearest a path extending along a second plurality of intersections between a second plurality of horizontal lines and a second plurality of vertical lines, wherein the second plurality of vertical lines are rotated through the correction angle relative to the first plurality of vertical lines; and

reading the pixel value data from the data buffer in the locations identified by the sequence of addresses to form the stabilized video signal.

### BRIEF DESCRIPTION OF THE DRAWINGS

FIG. 1 is a schematic view of apparatus for sensing the rotational orientation of a camera unit, built in accordance with a first embodiment of the invention;

FIG. 2 is a list of equations deriving an algorithm for interpreting the output of sensors within the apparatus of FIG. 1;

FIG. 3 is a table of values for a correction factor applied within the equations of FIG. 2;

FIG. 4 is a block diagram of an endoscopic image stabilization circuit built in accordance with the first embodiment of the invention to include the sensing apparatus of FIG. 1;

FIG. 5 is a block diagram of an alternate sensor board for use within the circuit of FIG. 4;

FIG. 6 is a flow chart showing a subroutine executing in a microcontroller within the circuit of FIG. 4 to generate angular correction data using an algorithm according to the equations of FIG. 2;

FIG. 7 is a pictographic view of the transformation of an image within the circuit of FIG. 4;

FIG. 8 is a flow chart showing a routine executing in an image processor within the image stabilization circuit of FIG. 4 in accordance with the first embodiment of the invention to perform the image transformation of FIG. 7;

FIG. 9 is a schematic view of apparatus for sensing the rotational orientation of a camera unit, built in accordance with a second embodiment of the invention;

FIG. 10 is a block diagram of a sensor board built in accordance with the second embodiment of the invention for use within the image stabilization circuit of FIG. 4; and

FIG. 11 is a flow chart showing a routine executing in an image processor within the image stabilization circuit of FIG. 4 including the sensor board of FIG. 10 in accordance with the second embodiment of the invention.

### DETAILED DESCRIPTION OF THE INVENTION

FIG. 1 is a schematic view of apparatus 10 for sensing rotational orientation built in accordance with a first embodiment of the invention to include an X-axis sensor 12 and a Y-axis sensor 14, oriented perpendicularly to one another in an x-y plane perpendicular to an axis of image rotation 16 of optics 18 within a video camera unit 20, which is focused on an image sensor 22 to form a video image within an endoscope. The axis of image rotation 16 is defined as an axis about which rotation of the camera unit 20 causes rotation of an image formed from an output signal produced in the image sensor 22. The camera unit 12 may be mounted to rotate within the endoscope about the axis of image rotation 16, or the camera unit 12 and the endoscope together may be rotated, deliberately or inadvertently within the body cavity to cause rotation of the camera unit about the axis of image rotation 16. If the camera unit 20 is mounted to rotate within the endoscope, the axis of such rotation is preferably aligned with an optical axis of the camera optics 18 and with the axis of image rotation 16 as defined herein, so that a process of correcting the image rotation will no cause a displayed image to wobble.

The direction sensors 12, 14 are rigidly attached to rotate in any direction with the camera optics 18 and with the image sensor 22. The camera optics 18 or elements within the camera optics 18 may be moved along the axis of image rotation 16 to change the magnification of the image directed to the image sensor 20. Each of the sensors 12, 14 is of a type that measures the strength of an ambient field, such as a magnetic field or a gravitational field. For example, during the use of the camera 20, an ambient magnetic field may be formed by the magnetic field of the earth or by an additional source of magnetism in combination with the earth's magnetic field. Alternately, a gravitational field is used. An ambient field of this kind is strongest in a direction that is understood to be the direction of the ambient field, and has a measured strength that is reduced as it is measured in directions rotated away from the direction of the field, reaching zero strength when measured at an angle perpendicular to the direction of the field.

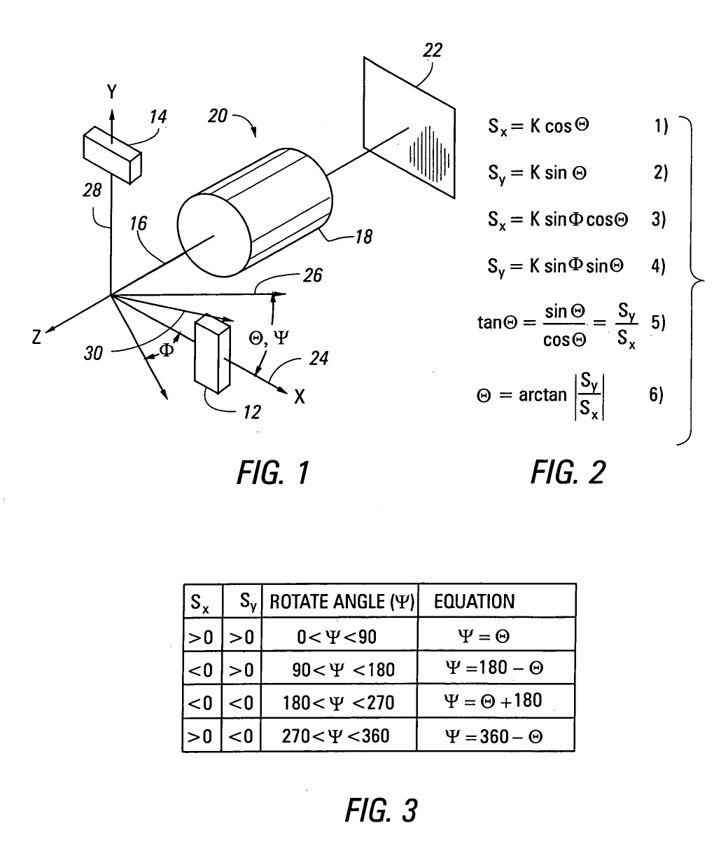

FIG. 2 is a list of equations deriving an algorithm for interpreting the outputs of the x-axis sensor 12 and the y-axis sensor 14. The x-axis sensor 12 is arranged to measure the field strength in the direction of the x-axis 14 (i.e., along the x-axis 24) of the camera 20, producing a signal Sₓ given by Equation 1, where Θ is the angle of rotation between the x-axis 24 and the direction 26 of the ambient field, in which the field is strongest, and K is a constant describing Sₓ when the field is aligned for maximum strength along the x-axis 24. Similarly, the y-axis sensor 14 is arranged to measure the field strength in the direction of the y-axis 28 (i.e. along the y-axis 28) of the camera 20, producing a signal S_{y}, given by Equation 2. If the direction of the ambient field (i.e. the direction of the in which the field is strongest) is additionally tilted through an angle φ from the plane formed by the x-axis 24 and the y-axis 28 into a direction 30, the strength of the signal from each of the sensors 12, 14 is further attenuated by a factor proportional to the sine of the angle φ, which is understood to by the acute angle formed between the direction 30 and the plane formed by the axes 24, 28, so that Sₓ and S_{y} are given by Equations 3) and 4), respectively. Since tilting the angle in which the field is strongest has the same attenuating effect on the signals from both sensors 12, 14, the tangent of the angle of rotation Θ is given by Equation 6), with Θ) being given by Equation 7).

In FIG. 2, the angle of rotation Θ is shown as an acute angle, having a value between 0 and 90 degrees. In general, the angle of rotation may be any angle from 0 degrees to 360 degrees. The value of the ratio S_{y}/Sₓ may be positive or negative, but this is not sufficient to locate the rotate angle between 0 degrees and 360 degrees. Therefore the signs of both Sₓ and S_{y} are considered in calculating an angle of rotation ψ, with the angle Θ from Equation 6) being considered a reference angle that is always between 0 and 90 degrees, being calculated from the absolute value of the ratio S_{y}/Sₓ.

FIG. 3 is a table showing the equations used to calculate the rotate angle ψ as a function of the reference angle Θ, the sign of the S_{y}, and the sign of Sₓ. For example, when the values of Sₓ and S_{y} are both positive, the rotate angle ψ is known to be between 0 and 90 degrees and equal to the reference angle Θ. If the value of Sₓ is negative, while the value of and S_{y} is positive, the rotate angle ψ is known to be between 90 and 180 degrees, with ψ being the difference between 180 degrees and the reference angle Θ. If the values of Sₓ and S_{y} are both negative, the rotate angle ψ is known to be between 180 and 270 degrees, with ψ being the sum of 180 degrees and the reference angle Θ. If the value of Sₓ is positive, while the value of S_{y} is negative, the rotate angle ψ is known to be between 270 degrees and 360 degrees, with ψ being given by the difference between 360 degrees and the reference angle Θ.

Thus, using calculations performed as described in reference to FIGS. 2 and 3, data from the direction sensors 12, 14 is used to determine the value of the angle of rotation ψ, between the x-axis 24 and a direction 26 within a plane perpendicular to the axis of image rotation 16 in which the ambient field is measured at its maximum level. The tangent of the angle of rotation is found by dividing a value of a signal from the y-axis sensor 14 by a value of the signal from the x-axis sensor 12, and the quadrant of the angle is found as a function of the signs of the values from the sensors 12, 14.

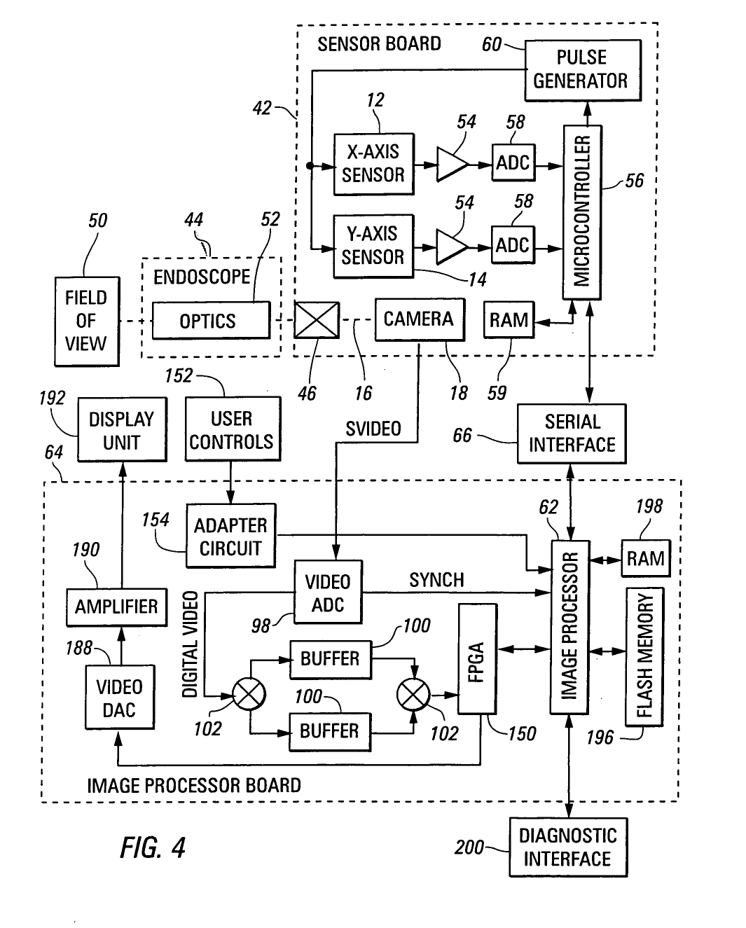

FIG. 4 is a block diagram of an image correction circuit 40 built in accordance with the with the first embodiment of the invention to include the sensing apparatus discussed above in reference to FIG. 1, with the x-axis sensor 12 and the y-axis sensor 14 being mounted, directly or indirectly, on a sensor circuit board 42, to which the camera unit 18 is additionally attached. Optionally, the sensor circuit board 42 and the camera unit 18 are also attached to the endoscope 44 through a bearing structure 46, allowing the rotation of the camera unit 18 and the sensor board 42 about the axis of image rotation 16 of the camera unit 18. Even if the camera unit 18 and the circuit board 42 are rigidly attached to the endoscope 44, rotation may occur as portions of the endoscope 44 are moved within the body cavity. The camera unit 18 views objects within a field of view 50 through endoscope optics 52. Since the sensors 12, 14 rotate with the camera 18, their rotation, relative to a fixed ambient field, such as gravity or the magnetic field of the earth, with the output signals from the sensors 12, 14 being used to determine the angular rotation of the cameral 18 about the axis of image rotation 16.

In a first version of the first embodiment of the invention, the sensors 12, 14 are built to measure the strength of the ambient magnetic field, with individual magnetostrictive sensing elements within a Wheatstone bridge providing a signal that is sensed as a bridge imbalance, being amplified through an instrumentation amplifier 54 associated with each of the sensors 12, 14. The output of each instrumentation amplifier 54 is provided as an input to a microcontroller 56 through an analog-to-digital converter 58, which is used to control the sensor system. The microcontroller 56 is provided with a random-access-memory 59 for storing data and program instructions. A high-current pulse generator 60, operating under control of the microcontroller 56, provides a series of pulses that are used to erase any magnetic bias within the sensors 12, 14. The microcontroller 56 on the sensor circuit board 42 is connected to the image processor 62 on the image processor circuit board 64 through a serial interface 66. The image processor 62, which is a microprocessor, performs rotations of the image to be displayed, based on camera angle data transmitted from the microcontroller 56.

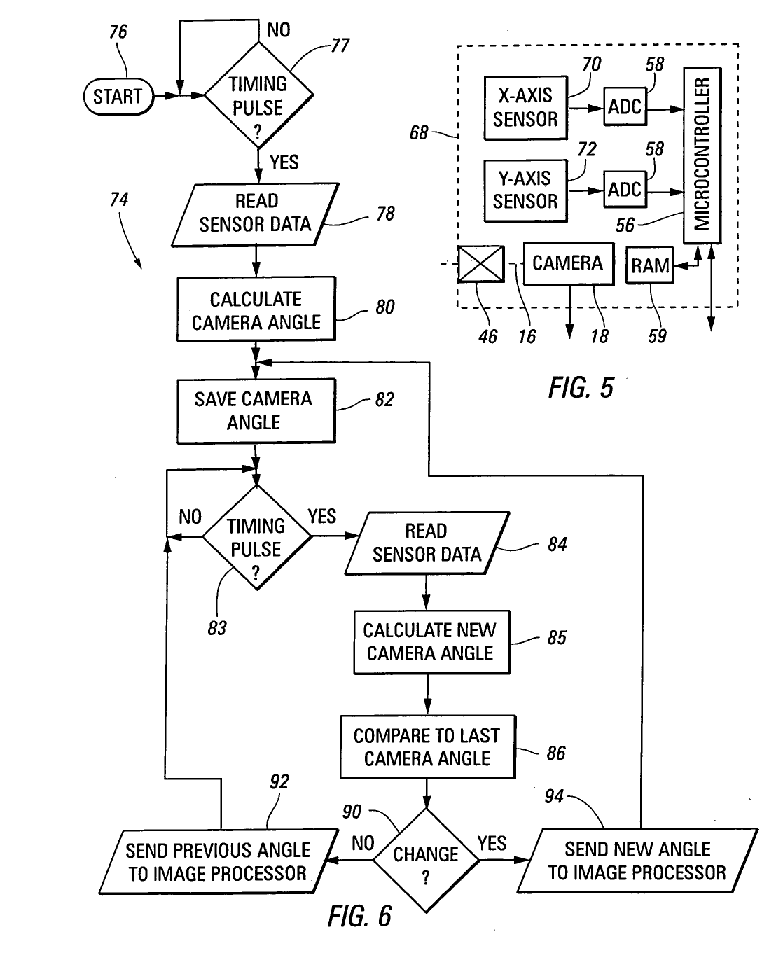

FIG. 5 is a block diagram of a sensor board 68 within a second version of the first embodiment of the invention, in which an x-axis sensor 70 and a y-axis sensor 72 in the form of accelerometers are provided to measure the angular position of the camera 18 relative to the direction of the ambient gravitational field. These sensors 70, 72 are mounted in the manner of sensors 12, 14, as described above in reference to FIG. 1. The outputs of sensors 70, 72 are provided to the microcontroller 56 through analog-to-digital converters 58. In FIGS. 5 and 6, similar elements are accorded like reference numerals.

FIG. 6 is a flow chart showing a subroutine 74 executing within the microcontroller 56 to provide output data specifying the angle of camera rotation. After starting in step 76, the subroutine 74 proceeds to step 77 to wait for a timing pulse indicating that it is time to read the sensors 12, 14 (or, alternately, sensors 70, 72). When this timing pulse occurs, data is read from the sensors in step 78. Then, the angle of camera rotation is calculated in step 80, using the method described above in reference to FIGS. 2 and 3, with the calculated angle of camera rotation being stored in step 82. Then, the subroutine 74 proceeds to step 83 to wait for the next timing pulse, which indicates that it is again time to read the sensors 12, 14 or 70, 72. When this timing pulse occurs, data from sensors is then read in step 84, with a new angle of camera rotation then being calculated in step 85. Next, in step 86, the new angle calculated in step 85 is compared with the angle saved in step 82. If it is determined in step 90 that the angle of camera rotation has not changed, the angle previously stored is sent to the image processor 62 in step 92, with the subroutine 74 then returning to step 83 to wait for the next time at which the sensor data is read. If the angle has changed, the new angle is sent to the image processor 62 in step 94, with the subroutine 74 then returning to step 82 to store the most recently read angle of camera rotation before proceeding to step 83.

Referring again to FIG. 4, the camera unit 18 produces an SVIDEO output signal, which is provided as an input to a video analog-to-digital converter 98 on the image processor circuit board 64, with a synchronization signal derived from the video signal input being provided as an input to the image processor 62, and with the digital video output data from the analog-to-digital converter 98 being written to one of two full-frame data buffers 100. The data buffers 100 are connected by switching circuits 102, which are operated so that data is written to one of the data buffers 100 while the data previously stored in the other data buffer 100 is being read.

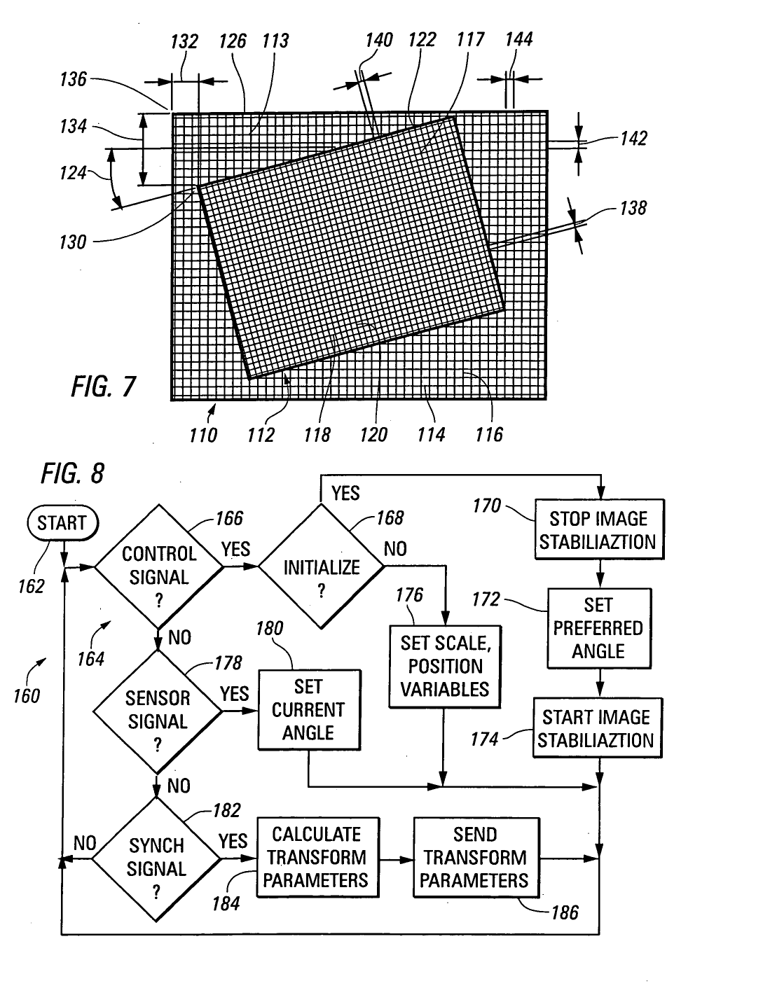

FIG. 7 is a pictographic view of the transformation of a digital image in accordance with the invention, within the apparatus of FIG. 4, with an input image 110, derived from output signal of the camera unit 12, formed by data written into one of the data buffers 100, being transformed into an output image 112. The input image 110 is formed by pixel values representing illumination intensities measured at points of intersection 113 between horizontal lines 114 and vertical lines 116. For example each of these pixel points 113 is associated with a single pixel value to form a gray-scale image or with three pixel values to form a color image. The output image 112 is formed by pixel values placed at points of intersection 117 between horizontal lines 118 and vertical lines 120, with a horizontal edge 122 of the output image 112 being rotated through a correction angle 124 from the corresponding horizontal edge 126 of the input image 110, and with a vertical edge 128 of the output image 112 being rotated through the same correction angle 124. A corner 130 of the output image 112 is translated through a horizontal distance 132 and through a vertical distance 134 from the corresponding corner 136 of the input image 110. Preferably, the output image 112 is formed by pixel values formed at the intersections of the same number of horizontal and vertical lines as the input image 110, with the distance 138 between adjacent horizontal lines 118 and the distance 140 between adjacent vertical lines 120 of the output image 112 being determined by multiplying the corresponding distances 142, 144 of the input image 110 by a scaling factor derived from the desired magnification of the input image 110.

In accordance with the invention, the correction angle 124 is calculated to hold the image at an orientation established during an initialization period, with a preferred angle being calculated using method described above in reference to FIGS. 1-3. Thus, the correction angle 124 is derived from the difference between the angle presently measured using this method and the preferred angle.

A field programmable grid array (FPGA) device 150 is used to perform the rotation of the image under control of the image processor 62, according to input signals received from the sensor board 62 to indicate the present orientation of the camera 18, and additionally according to input signals from the user controls 152, which cause control signals to be provided to the image processor 62 through an adapter circuit 154. In addition, the user controls 152 may be used to position the image to be displayed, varying the distances 124, 132, and to zoom the image, changing the magnification, and therefore the scaling factor used to calculate the distances 138, 140 between the lines at which pixel data will be displayed in the image.

FIG. 8 is a flow chart showing a process occurring within the image processor 62 during the execution of a routine 160 therein. After starting in step 162, the routine 160 enters a loop 164 to determine when a signal is received causing the image processor 62 to execute instructions for particular process steps.

For example, if it is determined in step 166 that a control signal has been received from the adapter circuit 154, indicating operation of the user controls 152, a further determination is made in step 168 of whether an attempt is being made to initialize the system, with an initialization process being provided to allow a preferred angle of rotation, at which the displayed image will subsequently be held, to be set. Thus, when it is determined in step 168 that the initialization process is being started, the process of stabilizing the image is stopped in step 170, so that a preferred angle of rotation can be established by manipulating the camera 18. When this process has been completed, the current angle, measured by the output from the sensor board 42 through the serial interface 66, is set as the preferred angle of rotation in step 172, and the process of image stabilization is begun in step 174. For example, the user may indicate that the initialization process should be started by depressing a first button in the user controls 152 and that the preferred angle has been found, so that the initialization process should be ended, by depressing a second button therein. This process is naturally used at the beginning of a session using the camera 18, with the image stabilization process not being started until step 174.

If it is determined in step 168 that the initialization process is not being started, other variables are set in step 176. For example, the user can use a zoom control within the user controls 152 to change the magnification, with the scaling variable used to determine the distances 138, 140 between the lines 118, 120 in the output image area 112 then being set. Alternately, the user can use position controls within the user controls 152 to establish the position of the displayed image, with values being set for the distances 132, 134.

If it is determined in step 178 of the loop 164 that a sensor signal has been sent through the serial interface 66 from the sensor board 42, a new value is set for the current angle in step 180.

If it is determined in step 182 of the loop 164 that a synch signal has been sent from the video analog-to-digital converter 98, parameters to be used within the FPGA device 150 for transforming the camera image 110 into the image 112 to be displayed are calculated in step 184. The correction angle 124 is calculated as a difference between the preferred angle last set in step 172 and the current angle last set in step 180. The scaling factor used to establish the distances 138, 140 between lines within the image 112 to be displayed, and the image displacement distances 134, 136 are calculated according to variables last set in step 176. These transform parameters, which are generated in a form used by algorithms executing within the FPGA device 150 to perform the image transformation, are sent to the FPGA device 150 in step 186.

Referring again to FIGS. 6 and 7, within the FPGA device 150, image transform parameters sent from the image processor 62 are used to generate, for each horizontal line 118 within the output image 112, a sequence of address locations, with each of the address locations representing, for a sequence of intersection points 117 between the line 118 and the vertical lines 120 of the output image, an address within the input image 110 representing the closest intersection point 113 between the horizontal lines 114 and the vertical lines 116. For each of the horizontal lines 118 within the output image 112, data stored within the data buffer 100 currently being read is read according to the sequence of address locations generated in this way. The data thus developed for each of the horizontal lines 118 is read in the sequence of the horizontal lines 118, so that data is read from the data buffer 100 in the form of a raster pattern forming the output image, with this data being sent as a digital video signal to a video digital-to-analog converter 188. The output signal from the video digital-to-analog converter 188 is provided as a stabilized video signal input to a video amplifier 192, which produces a stabilized video signal driving a display unit 194.

Thus, a portion of the pixel value data from the input frame, that has been recorded at storage locations corresponding to points along a first path, such as a raster pattern formed to include horizontal lines 114of the input image 110, is read according to a sequence of storage locations corresponding to points along a second path, such as a raster pattern formed to include horizontal lines 118 of the output image 112. If the output image 112 extends outside the input image 110, the pixel positions within the output image 112 that cannot be filled with data from the input image 110 are sent in a form resulting in a black local image display.

Instructions for routines to be executed within the image processor 62 and the microcontroller 56 are stored within a flash memory 196, to be loaded upon system start-up. The image processor 62 is additionally provided with a random access memory 198 for storing data and program instructions. Optionally, a diagnostic serial interface 200 may also be provided for connection to an external diagnostic device.

During operation of the apparatus in accordance with the first embodiment of the invention, as described above, image stabilization is accomplished by tracking a current angle of rotation, within a plane of rotation perpendicular to the axis of camera rotation, of the camera image sensor relative to the direction of the ambient field, and by then transforming the image from the camera to compensate for the rotation of the edge of the image. This current angle of rotation is tracked by measuring values of the field in directions perpendicular to the axis of image rotation and perpendicular to one another. A limitation of this process results from the fact that, if the axis of image rotation is moved parallel to the direction of the ambient field, rotation of the camera unit will have no effect on the levels of the field being measured, so the angle of camera rotation cannot be tracked. For example, when the ambient gravitational field is being measured, the method will not work if the camera unit is pointed straight downward or straight upward, with the axis of image rotation being moved to a vertical axis. As a practical matter, the accuracy with which the current angle of camera rotation can be measured is reduced as the condition is approached in which the axis of image rotation is parallel the direction of the ambient field. This limitation is avoided by using an apparatus built in accordance with a second embodiment of the invention, in which two sets of sensors are used to sense two different ambient fields that occur in different directions. For example, one set of sensors measures the ambient magnetic field, while the other set of sensors measures the ambient gravitational field.

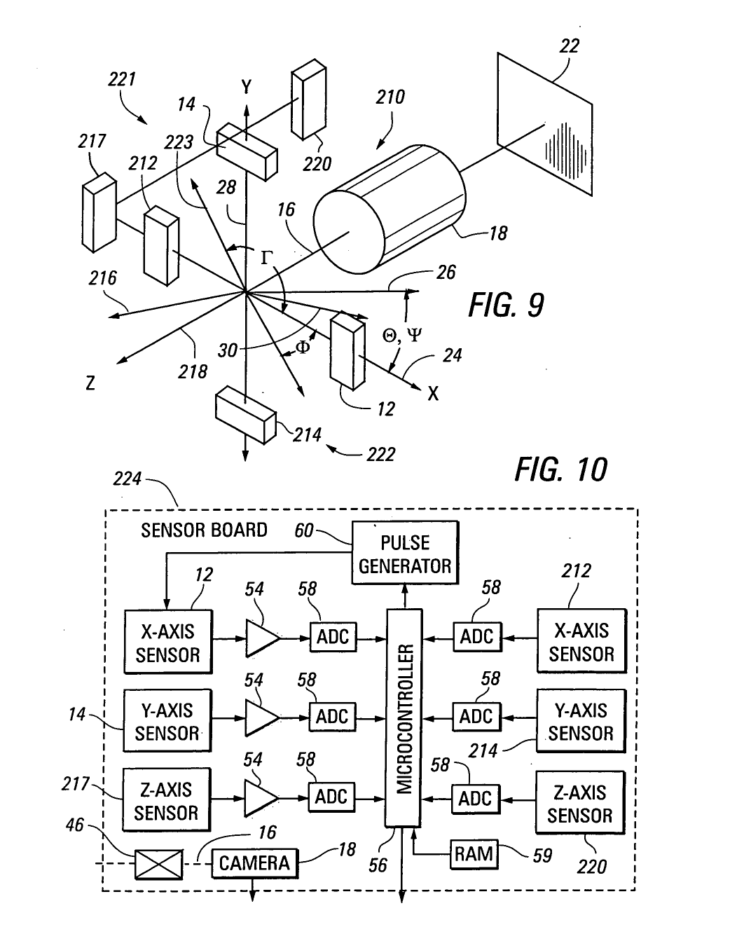

FIG. 9 is a schematic view of apparatus 210 built in accordance with a second embodiment of the invention to include a first pair of direction sensors 12, 14, mounted as described above in reference to FIG. 1 to determine the angular relationship of the camera unit 18 relative to a first ambient field acting in a direction indicated by arrow 30, and additionally to include a second pair of direction sensors 212, 214. Like the direction sensors 12, the direction sensors 212, 214 are mounted so that the directions in which they sense an ambient field are perpendicular to one another, and so that these directions of field sensing are additionally perpendicular to the axis of image rotation 16 of the camera unit 18. The additional direction sensors 212, 214 sense another type of ambient field, extending in a different direction, as indicated by arrow 216.

Optionally, an first z-axis sensor 217, sensing a component of the first ambient field in the Z-direction 218, and a second z-axis sensor 220, sensing a component of the second ambient field in the z-direction 218, which is parallel to the axis of image rotation, may be provided to obtain data regarding camera movement in a tilt direction or regarding the effect of an ambient field in a plane extending in the z-axis direction 218. It is noted that any of the sensors may be displaced from the x-, y-, or z-axis with which they are associated, as long as they are mounted on a rigid structure moving with the camera unit 18.

Preferably, a first sensing unit 221, including direction sensors 12, 14 and a second second sensing unit 222, including direction sensors 212, 214 are separately and alternately used, with data from the first pair of direction sensors 12, 14 being used to determine the angle θ, between the x-axis 24 and the direction 26, within a plane perpendicular to the axis of image rotation 16, in which the first ambient field is measured at its maximum level, and with data from the second pair of direction sensors 212, 214 being used to determine the angle ┌ between the x-axis 24 and the direction 223, also within a plane perpendicular to the axis of image rotation 16, in which the second ambient field is measure at its maximum level. The calculations discussed above in reference to FIGS. 2 and 3 are separately applied to data from the first pair of direction sensors 12,14 and to data from the second pair of direction sensors 212, 214.

While sensors 12, 212 are shown as sensing ambient fields in the same direction, that of the x-axis 24, and while sensors 14, 214 are shown as sensing ambient fields in the same direction, that of the y-axis 28, it is only necessary that the sensors 12, 14 must sense the first ambient field in directions perpendicular to one another and to the axis of image rotation 16, and that the sensors 121, 214 must sense the second ambient field in directions perpendicular to one another and to the axis of image rotation 16.

FIG. 10 is a block diagram of a sensor circuit board 224 built in accordance with the second embodiment of the invention, including sensors mounted as shown in FIG. 9, with elements similar to those shown in FIG. 3 being accorded like reference numbers. For example, each of the sensors 12, 14, 217 is a device measuring the strength of the ambient magnetic field, with individual magnetostrictive sensing elements within a Wheatstone bridge providing a signal that is sensed as a bridge imbalance, being amplified through an instrumentation amplifier 54 and converted into a digital signal within an analog-to-digital converter 58 to provide an input to the microcontroller 56. A high-current pulse generator 60, operating under control of the microcontroller 56, provides a series of pulses that are used to erase any magnetic bias within the sensors 12, 14. Each of the sensors 212, 214, 220 is an accelerometer measuring the effect of ambient gravity in a certain direction, producing an analog signal that is converted into a digital signal in an analog-to-digital converter 58 to be provided as an input to the microcontroller 56. In accordance with the second embodiment of the invention, the sensor circuit board 224 replaces the sensor circuit board 42 within the image stabilization circuit of FIG. 4, with the microcontroller 56 on the sensor circuit board 224 being connected to the image processor 62 on the image processor circuit board 64 through the serial interface 66.

The method of the second embodiment of the invention provides an ability to choose between the use of two ambient fields, such as gravity and the ambient magnetic field. In a first version of the second embodiment, the user controls 152 (shown in FIG. 4) are provided with a means to switch between using the two ambient fields. Such a determination may be made to avoid a situation in which the accuracy of the image stabilization process will be compromised by the orientation of the optical axis of the camera unit. For example, the gravitational field may be used except when the optical axis is nearly vertical, with the ambient magnetic field then being used to maintain accuracy.

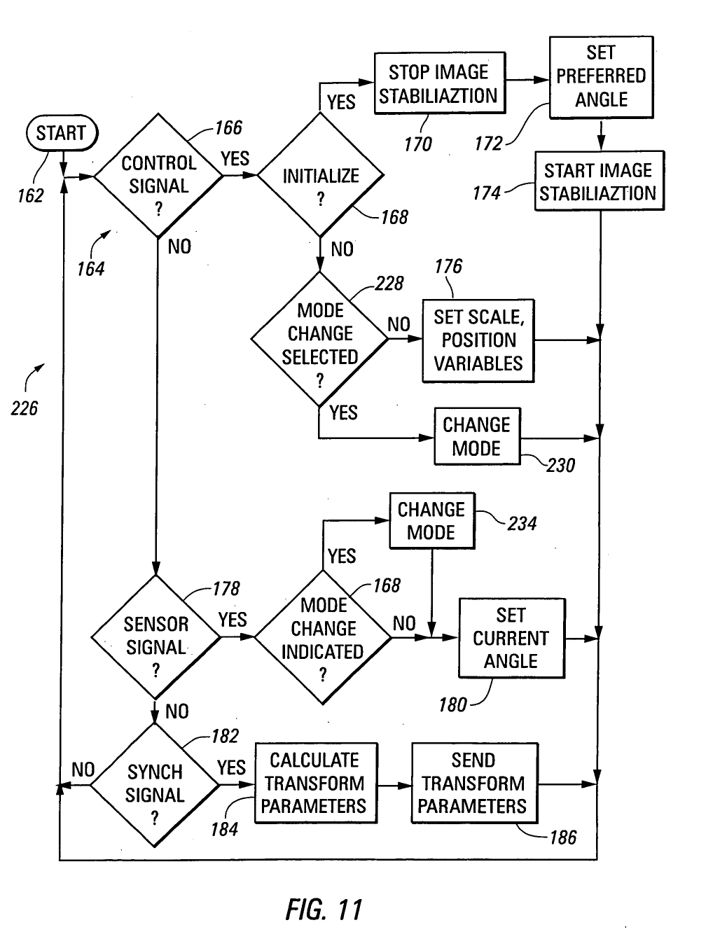

FIG. 11 is a flow chart showing a routine 226 executing within the image processor 62 (shown in FIG. 4), providing for operation in accordance with the second embodiment of the invention with the sensor board 224, described above in reference to FIG. 10. The routine 226 includes many process steps that are similar or identical to process steps within the routine 160, which has been explained above in reference to FIG. 8. Such similar process steps, which are accorded like reference numbers with process steps from FIG. 8, are understood to operate as described above, and will not be discussed in detail again.

In accordance with the second embodiment of the invention, the user controls 152 (shown in FIG. 4) include a mode control, which can be used, for example, to choose between a magnetic field mode, in which the ambient magnetic field sensed by sensors 12, 14 is used for image stabilization, a gravitational field mode, in which the gravitational field sensed by sensors 212, 214 is used for image stabilization, and a automatic mode, in which the apparatus determines which field should be used for image stabilization. Thus, when it is determined in step 168 that a control signal sent from the adapter circuit 154 in response to operation of the user controls 152 does not call for initialization, the routine 226 proceeds to step 228, in which a further determination is made of whether a mode change has been requested. If it has, the mode change is made in step 230.

When the apparatus is operating in the automatic mode, a mode change may additionally be indicated by the levels of sensor signals read in step 178. For example, since the levels of both the X-axis sensor being currently used and the Y-axis sensor being currently used decrease as the direction in which the ambient field being currently used approaches the axis of image rotation 16, in a first version of the second embodiment of the invention, it is determined in step 232 that a mode change is indicated as being desirable when the sum of the signal levels from the X-axis sensor being currently used and the Y-axis sensor being currently used falls below a predetermined threshold level. For example, referring additionally to FIG. 6, a summation value representing the sum of the signal levels from the x-axis sensor and from the y-axis sensor is calculated along with the new camera angle in step 85 of the subroutine 74 executing within the microcontroller 56, with this summation value being sent to the image processor in step 92 or in step 94, since it is possible that this summation value could change without a change in the angle of camera rotation. Then, in step 232, this summation value, which has been received as a sensor signal from the sensor circuit board 224, is compared with the threshold level to determine if a mode change is indicated by this summation value being too low.

In a second version of the second embodiment of the invention, the optional z-axis sensors 217, 220 are included, with their signals being used to determine, in step 232, that a mode change is indicated as being desirable. In this regard, it is noted that, as the axis of rotation approaches the direction in which the ambient field is maximized, the absolute value of the signal level from the z-axis sensor increases. Thus, in steps 92 and 94 of the subroutine 74 (shown in FIG. 6) a value corresponding to the signal level from the z-axis sensor 216, 220 being currently used is transmitted along with the angle of camera rotation. Then, in step 232, this value corresponding to the z-axis signal level is compared with a threshold level to determine if a mode change is indicated by this value being too great. The signals from the z-axis sensors 217, 220 may also be used, together with signals from the other sensors, to establish tilt angle data, generally as described above in reference to FIGS. 2 and 3.

In either version of the second embodiment of the invention, when it is determined in step 232 that a mode change is required, the mode is changed in step 234. For example, the apparatus may begin operating with measurements of the camera orientation relative to the gravitational field, and then, as the axis of image rotation 16 is moved close to a vertical direction, switch to operate with measurements of the camera orientation relative to the ambient magnetic field. Then, as the axis or image rotation 16 is moved close to the angle in which the absolute value of the ambient magnetic field is measured at a maximum level, the apparatus switches back to using the gravitational field.

Referring additionally to FIG. 9, In the step 172 of the routine 226, the preferred angle may be stored as both the angle ψ of the camera unit relative to the direction in which the absolute value of the first ambient field is maximized and as the angle ┌ of the camera unit relative to the direction in which the absolute value of the second ambient field is maximized. Alternately, in step 172, the preferred angle may be stored only as the angle relative to the direction of the ambient field used within the current mode, with a new preferred angle being set as the angle relative to the direction of the other ambient field when the mode is changed in step 230, 234. This can be done because the camera unit is held at the preferred angle, which can be measured with either set of sensors, before the mode is changed.

The second embodiment of the invention is alternately implemented without the automatic mode changing process described above as occurring during steps 232, 234, with mode changing only occurring in response to operation of the user controls 152, or with the apparatus being set to operate in one mode or the other before operation is begun.

While the invention has been described in terms of preferred embodiments and versions with some degree of particularity, it is understood that this description has been given only by way of example, and that many changes can be made without departing from the spirit and scope of the invention, as defined in the appended claims.

What is claimed is:

1. A method for stabilizing a displayed image formed from an output signal of a camera unit used with an endoscope, wherein
the method comprises periodically determining a value of an angle of rotation of the camera unit about an axis of image rotation relative to a direction of an ambient field by an angle determining process comprising:
receiving a first signal representing a level of the ambient field measured in a first direction relative to the camera unit, wherein the first direction is perpendicular to the axis of image rotation;
receiving a second signal representing a level of the ambient field measured in a second direction relative the camera unit, wherein the second direction is perpendicular to the axis of image rotation and to the first direction;
determining a tangent of the angle of rotation by dividing a value representing a level of the first signal by a value representing a level of the second signal; and
determining a quadrant of the angle of rotation as a function of signs of the first and second signals,
rotation of the camera unit about the axis of image rotation causes rotation of an image formed from the output signal of the camera unit,
a value of the angle of rotation is set as a preferred angle of rotation,
the output signal of the camera unit is processed to form a stabilized video signal by an image stabilizing process causing the displayed image to be rotated through a correction angle determined by calculating a difference between the most recently determined value of the angle of rotation of the camera unit and the preferred angle of rotation, and
a display unit is driven with the stabilized video signal to form the displayed image.

2. The method of claim 1, wherein the ambient field is a magnetic field.

3. The method of claim 2, wherein
the first signal is generated from an imbalance in a first Wheatstone bridge including a magnetostrictive element sensing a magnetic field in the first direction, and
the second signal is generated from an imbalance in a second Wheatstone bridge including a magnetostrictive element sensing a magnetic field in the second direction.

4. The method of claim 1, wherein the ambient field is a gravitational field.

5. The method of claim 4, wherein
the first signal is generated from an output signal of an accelerometer sensing a component of the gravitational field in the first direction, and
the second signal is generated from an output signal of an accelerometer sensing a component of the gravitational field in the second direction.

6. The method of claim 1, wherein the image stabilizing process comprises:
writing pixel value data derived from the output signal of the camera unit, representing light intensities measured at an image sensor within the camera unit at a first plurality of intersections between a first plurality of horizontal lines and a first plurality of vertical lines to a corresponding plurality of locations within a data buffer;
generating a sequence of addresses identifying locations within the corresponding plurality of locations within the data butter, wherein the sequence of addresses identifies locations storing data representing light intensities measured nearest a path extending along a second plurality of intersections between a second plurality of horizontal lines and a second plurality of vertical lines, wherein the second plurality of horizontal lines are rotated through a correction angle relative to the first plurality of horizontal lines, wherein the second plurality of vertical lines are rotated through the correction angle relative to the first plurality of vertical line; and
reading the pixel value data from the data buffer in the locations identified by the sequence of addresses to form the stabilized video signal.

7. A method for stabilizing a displayed image formed from an output signal of a camera unit used with an endoscope, wherein
the method comprises periodically determining a value of an angle of rotation of the camera unit about an axis of image rotation relative to a direction of an ambient field,
rotation of the camera unit about the axis of rotation causes rotation of and image formed from the output signal of the camera unit,
a value of the camera unit is set as a preferred angle of rotation,
the output signal of the camera unit is processed to form a stabilized video signal by an image stabilizing process causing the displayed image to be rotated through a correction angle determined by calculating a difference between the most recently determined value of the angle of rotation of the camera unit and the preferred angle of rotation,
the image stabilizing process comprises:
writing pixel value data derived from the output signal of the camera unit, representing light intensities measured at an image sensor within the camera unit at a first plurality of intersections between a first plurality of horizontal lines and a first plurality of vertical lines to a corresponding plurality of locations within a data buffer;
generating a sequence of addresses identifying locations within the corresponding plurality of locations within the data butter, wherein the sequence of addresses identifies locations storing data representing light intensities measured nearest a path extending along a second plurality of intersections between a second plurality of horizontal lines and a second plurality of vertical lines, wherein the second plurality of horizontal lines are rotated through a correction angle relative to the first plurality of horizontal lines, wherein the second plurality of vertical lines are rotated through the correction angle relative to the first plurality of vertical line; and
reading the pixel value data from the data buffer in the locations identified by the sequence of addresses to form the stabilized video signal; and
a display unit is driven with the stabilized video signal to form the displayed image.

8. The method of claim 7, wherein
the data buffer comprises first and second data buffer areas,
the pixel value data is alternately written to locations within the first and second data buffer areas,
data is read from locations within the second data buffer area as the pixel value data is written to locations within the first data buffer area, and
data is read from locations within the first data buffer area as the pixel value data is written to locations within the second data buffer area.

9. A method for stabilizing a displayed image formed from an output signal of a camera unit used with an endoscope, wherein
rotation of the camera unit about an axis of image rotation causes rotation of an image formed from the output signal of the camera unit,
the method comprises periodically determining a value of an angle of rotation of the camera unit about the axis of image rotation by an angle determining process comprising;
determining whether the axis of image rotation is spaced away from a direction of a first ambient field through an angle sufficient to allow accurate determination of the angle of rotation by determining the rotation of the camera unit relative to the direction of the first ambient field;
determining an angle of rotation of the camera unit relative to the direction of the first ambient field in response to determining that the axis of image rotation is spaced away from the direction of the first ambient field through an angle sufficient to allow accurate determination of the angle of rotation by determining the rotation of the camera unit relative to the first ambient field; and
determining an angle of rotation of the camera unit relative to the direction of a second ambient field in response to determining that the axis of image rotation is not spaced away from the direction of the first ambient field through an angle sufficient to allow accurate determination of the angle of rotation by determining the rotation of the camera unit relative to the first ambient field, and
a value of the angle of rotation is set as a preferred angle of rotation,
the output signal of the camera unit is processed to form a stabilized video signal by an image stabilizing process causing the displayed image to be rotated through a correction angle determined by calculating a difference between the most recently determined value of the angle of rotation of the camera unit and the preferred angle of rotation, and
a display unit is driven with the stabilized video signal to form the displayed image.

10. The method of claim 9, wherein a determination of whether the axis of image rotation is spaced away from a direction of a first ambient field through an angle sufficient to allow accurate determination of the angle of rotation by determining the rotation of the camera unit relative to the direction of the first ambient field comprises:
receiving a first signal representing a level of the first ambient field measured in a first direction relative to the camera unit, wherein the first direction is perpendicular to the axis of image rotation;
receiving a second signal representing a level of the first ambient field measured in a second direction relative the camera unit, wherein the second direction is perpendicular to the axis of image rotation and to the first direction; and
determining whether a sum of levels of the first and second signals exceeds a predetermined value.

11. The method of claim 10, wherein
a determination of the angle of rotation of the camera unit relative to the direction of the first ambient field comprises:
determining a tangent of the angle of rotation of the camera unit relative to the first ambient field by dividing a value representing the level of the first signal by the value representing the level of the second signal; and
determining a quadrant of the angle of rotation of the camera unit relative to the first ambient field as a function of signs of the first and second signals; and
a determination of the angle of rotation of the camera unit relative to the direction of the second ambient field comprises:
receiving a third signal representing a level of the second ambient field measured in a third direction relative to the camera unit, wherein the third direction is perpendicular to the axis of image rotation;
receiving a fourth signal representing a level of the second ambient field measured in a fourth direction relative the camera unit, wherein the third direction is perpendicular to the axis of image rotation and to the second direction;
determining a tangent of the angle of rotation of the camera unit relative to the second ambient field by dividing a value representing the level of the third signal by the value representing the level of the fourth signal; and
determining a quadrant of the angle of rotation of the camera unit relative to the first ambient field as a function of signs of the third and fourth signals.

12. The method of claim 9, wherein a determination of whether the axis of image rotation is spaced away from a direction of a first ambient field through an angle sufficient to allow accurate determination of the angle of rotation by determining the rotation of the camera unit relative to the direction of the first ambient field comprises:
receiving a z-axis signal representing a level of the first ambient field measured in a direction parallel to the axis of image rotation; and
determining whether a level of the z-axis signal is less than a predetermined value.

13. The method of claim 12, wherein
a determination of the angle of rotation of the camera unit relative to the direction of the first ambient field comprises:
receiving a first signal representing a level of the first ambient field measured in a first direction relative to the camera unit, wherein the first direction is perpendicular to the axis of image rotation;
receiving a second signal representing a level of the first ambient field measured in a second direction relative the camera unit, wherein the second direction is perpendicular to the axis of image rotation and to the first direction;
determining a tangent of the angle of rotation of the camera unit relative to the first ambient field by dividing a value representing the level of the first signal by the value representing the level of the second signal; and
determining a quadrant of the angle of rotation of the camera unit relative to the first ambient field as a function of signs of the first and second signals; and
a determination of the angle of rotation of the camera unit relative to the direction of the second ambient field comprises:
receiving a third signal representing a level of the second ambient field measured in a third direction relative to the camera unit, wherein the third direction is perpendicular to the axis of image rotation;
receiving a fourth signal representing a level of the second ambient field measured in a fourth direction relative the camera unit, wherein the third direction is perpendicular to the axis of image rotation and to the second direction;
determining a tangent of the angle of rotation of the camera unit relative to the second ambient field by dividing a value representing the level of the third signal by the value representing the level of the fourth signal; and
determining a quadrant of the angle of rotation of the camera unit relative to the first ambient field as a function of signs of the third and fourth signals.

14. Apparatus for displaying a stabilized image comprising:
an endoscope;
a camera unit used with the endoscope, including an image sensor forming an output signal, wherein the camera unit is rotatable about an axis of image rotation, and wherein rotation of the camera unit about the axis of image rotation causes rotation of an image formed from the output signal;
a first field sensing device sensing an angle or rotation of the camera unit about the axis of image rotation relative to a direction of a first ambient field;
a second field sensing device sensing an angle of rotation of the camera unit abour the axis of image rotation relative to a direction of a second ambient field;
a processor, operable in a first mode using signals from the first sensing device and in a second mode using signals from the second field sensing device, periodically determining a value of an angle of rotation of the camera unit about the axis of image rotation, storing a value of the angle of rotation as a preferred angle of rotation, and calculating a correction angle as a difference between a most recently determined value of the angle of rotation and the preferred angle of rotation;
data storage; storing pixel value data representing the output signal from the camera unit, wherein the pixel value data is read from the data storage to form a stabilized video signal in a sequence causing an image formed from the stabilized video signal to be rotated through the correction angle;
a display unit driven by the stabilized video signal to display the stabilized video image.

15. The apparatus of claim 14, wherein the apparatus is additionally operable in an initialization mode for setting the preferred angle of rotation with the pixel video data being read from data storage in a sequence causing the image formed from the stabilized video signal to be displayed without rotation relative to an image from the output signal from the camera unit.

16. The apparatus of claim 14, wherein
the first field sensing device comprises a first sensor, generating a first signal representing a level of the first ambient field in a first direction, perpendicular to the axis of image rotation; and a second sensor, generating a second signal representing a level of the first ambient field in a second direction, perpendicular to the axis of image rotation and perpendicular to the first direction,
the second field sensing device comprises a third sensor, generating a third signal representing a level of the second ambient field in a third direction, perpendicular to the axis of image rotation, and a fourth sensor, generating a fourth signal representing a level of the second ambient field in a fourth direction, perpendicular to the axis of image rotation and to the third direction,
a tangent of the angle of rotation of the camera unit relative to the direction of the first ambient field is calculated by dividing a value of the first signal by a value of the second signal,
a quadrant of the angle of rotation of the camera unit relative to the direction of the first ambient field is determined as a function of a sign a value of of the first signal and a sign a value of the second signal,
a tangent of the angle of rotation of the camera unit relative to the direction of the second ambient field is calculated by dividing a value of the third signal by a value of the fourth signal, and
a quadrant of the angle of rotation of the camera unit relative to the direction of the second ambient field is determined as a function of a sign a value of of the third signal and a sign a value of the fourth signal,

17. The apparatus of claim 16, wherein the first ambient field is a magnetic field and the second ambient field is a gravitational field.

18. The apparatus of claim 14, additionally comprising a field programmable grid array device, wherein
the processor transmits data representing the correction angle to the field programmable grid array device,
the field programmable grid array device generates a sequence of addresses identifying locations within the data storage for reading data to form the stabilized video signal.

19. The apparatus of claim 14, additionally comprising a user control selectable to cause operation in the first mode and in the second mode.

20. The apparatus of claim 14, wherein
the processor, operating in the first mode, additionally determines whether the axis of image rotation is spaced away from the direction of the first ambient field through an angle sufficient to allow accurate determine of the angle of rotation by determining the direction of rotation of the camera unit relative to the direction of the first ambient field, and
the processor, operating in the second mode, additionally determines whether the axis of image rotation is spaced away from the direction of the second ambient field through an angle sufficient to allow accurate determine of the angle of rotation by determining the direction of rotation of the camera unit relative to the direction of the second ambient field.
